(19) United States
(12) Patent Application Publication
(10) Pub. No.: US 2011/0008878 A1
(43) Pub. Date: Jan. 13, 2011

(54) TARGET NUCLEIC ACID MEASURING APPARATUS

(75) Inventors: Yoshinobu Akimoto, Tokyo (JP); Mari Nakamoto, Tokyo (JP); Kaneyasu Ookawa, Sagamihara-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: OLYMPUS CORPORATION, Tokyo (JP)

(21) Appl. No.: 12/692,240

(22) Filed: Jan. 22, 2010

(30) Foreign Application Priority Data
Jul. 7, 2009 (JP) 2009-160974

(51) Int. Cl. C12M 1/34

(52) U.S. Cl. 435/287.2

(57) ABSTRACT

The present invention relates to calculation of an initial template amount in a test sample by fitting a theoretical expression to detection intensity of target nucleic acid corresponding to thermal cycle number, wherein the theoretical expression includes an environmental coefficient as an exponent parameter, a parameter of the initial template amount in the test sample, and terms for internal standard correction and baseline correction of the detection intensity of the amplification amount of the target nucleic acid, and includes at least one parameter among a saturation amount upon the target nucleic acid amplification, a reaction acceleration coefficient, and a reaction inhibition coefficient.

### CROSS-REFERENCE TO RELATED APPLICATIONS

This application is based upon and claims the benefit of priority from Japanese Patent Application No. 2009-160974, filed Jul. 7, 2009, the entire contents of which are incorporated herein by reference.

### BACKGROUND OF THE INVENTION

1. Field of the Invention

The present invention relates to a target nucleic acid measuring method, a target nucleic acid measuring apparatus, a target nucleic acid measuring system, and a target nucleic acid measuring program.

2. Description of the Related Art

There has been conventionally reported a nucleic acid amplification reaction called polymerase chain reaction (hereinafter, described as “PCR”). The PCR is capable of amplifying a specific nucleic acid region in a nucleic acid molecule, even to about one million times the original amount within a vessel. By utilizing the PCR, it is possible to detect a specific nucleic acid region of target nucleic acid in an amplified state, and in terms of sensitivity, it is even possible to detect a pathogen from the nucleic acid of one molecule.

As a general method for detecting this amplified specific nucleic acid region, there is available a method of separating the reaction solution obtained after completion of a PCR using agarose electrophoresis, subsequently staining the nucleic acids, and discriminating the specific nucleic acid region based on the degree of mobility of the band (molecular weight); or a method of detecting this reaction solution by a dot hybridization method.

Here, a method for measuring the initial template amount is disclosed in JP-A-7-163397. In this method of measurement, first, a plurality of test samples are prepared such that one test sample has a specific nucleic acid sequence at an unknown concentration, while other test samples contain the same specific nucleic acid sequence at different known concentrations. Then, the test samples of known concentrations and the test sample of unknown concentration are subjected to a thermal cycle concurrently in multiple cycles. Subsequently, the fluorescence radiated from the test samples is measured, and the number of cycles required for each of the reaction mixtures to emit fluorescence at a certain intensity (for example, a predetermined intensity at or above the detection level) in real time (hereinafter, referred to as “C_{T} value”) is determined. Then, a standard curve plotting the concentration of the specific nucleic acid sequence versus the C_{T} value is produced for the test samples of known concentrations, and the C_{T} value of the test sample of unknown nucleic acid concentration is assigned into the produced standard curve, to thereby determine the amount of initial template of the specific nucleic acid sequence in the test sample of unknown concentration.

The product manual of the Real-Time PCR System (trade name) manufactured by Applied Biosystems, Inc. discloses a comparative C_{T} method, which is a relative quantification method of determining a relative value from the difference between C_{T} values of a test sample and a reference sample (see URL: http://www.appliedbiosystems.co.jp/website/jp/biobeat/contents.jsp?COLUMNPGCD=78973&COLUMNCD=76448&TYPE=C&BIOCATEGORYCD=7). In this method, a relative value is determined from the difference between the C_{T} values of the test sample and the reference sample, and the initial template amount is determined under an assumption that the PCR amplification efficiency is 100%.

JP-A-8-66199 also discloses a method for measuring the initial template amount as follows. In this measuring method, first, a test sample is subjected to thermal cycles in a plurality of cycles. At this time, since fluorescence is radiated from the test sample at an intensity equivalent to the amplification amount of the nucleic acid, this radiated fluorescence is measured. Then, the measured value is converted to a molar concentration value of dsDNA, and a measurement curve plotting the molar concentration of dsDNA versus the cycle number is generated. A theoretical curve that takes the starting molar concentration of the primer as one of the parameters is fitted to the measurement curve, and thus the molar concentration of dsDNA at cycle number 0, that is, the initial template amount in the test sample, is determined.

### SUMMARY OF THE INVENTION

A target nucleic acid measuring apparatus according to one aspect of the present invention includes a storage unit and a control unit. The storage unit includes an amplification amount detection intensity storage unit that stores a detection intensity of an amplification amount of target nucleic acid corresponding to thermal cycle number, and a theoretical expression storage unit that stores a theoretical expression. The theoretical expression includes an environmental coefficient as an exponent parameter, a parameter of an initial template amount in the test sample, and terms for the internal standard correction and baseline correction of the detection intensity of the amplification amount of the target nucleic acid. The theoretical expression further includes at least one parameter among a saturation amount upon the target nucleic acid amplification, a reaction acceleration coefficient, and a reaction inhibition coefficient. The control unit includes a theoretical expression fitting unit that fits the theoretical expression to the detection intensity of the amplification amount of the target nucleic acid for each thermal cycle number stored in the storage unit. The control unit further includes an initial template amount calculating unit that calculates the initial template amount from the theoretical expression fitted by the theoretical expression fitting unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a flowchart for explaining an overview of the present embodiment;

FIG. 2 is a block diagram showing an example of a configuration of a target nucleic acid measuring apparatus 100 according to an embodiment to which the present invention is applied;

FIG. 3 is a flowchart showing an example of the processing of the target nucleic acid measuring apparatus 100 according to the present embodiment;

FIG. 4 is a diagram showing raw data (unprocessed measurement data) regarding detection intensity (luminance) of the amplification amount of the target sample (target nucleic acid) and that of the static internal standard, which are obtained by the measuring apparatus ABI7900 (trade name);

FIG. 5 is a diagram showing the luminance ratio data (I₁ₙ/I₀ₙ) of the target sample and the internal standard, calculated from the raw data shown in FIG. 4;

FIG. 6 is a diagram showing the data (I₁ₙ/I₀ₙ−1.675) obtained by subtracting the baseline 1.675 from the luminance ratio shown in FIG. 5;

FIG. 7 is a diagram showing the measurement result of FAM and ROX in the test sample;

FIG. 8 is a diagram showing the result of the fitting;

FIG. 9 is a diagram showing values of the parameters obtained by the fitting;

FIG. 10 is a diagram comparing the number of the prepared target nucleic acid copies, with the number of the target nucleic acid copies calculated in this Example 2; and

FIG. 11 is a diagram showing the relationship between the molar concentration of FAM molecules and the fluorescence intensity.

### DETAILED DESCRIPTION OF THE PREFERRED EMBODIMENTS

Hereinafter, exemplary embodiments of a target nucleic acid measuring method, a target nucleic acid measuring apparatus, a target nucleic acid measuring system, and a target nucleic acid measuring program according to the invention will be explained in detail with reference to the accompanying drawings. The invention is not limited to the embodiments.

### Overview of Present Embodiment

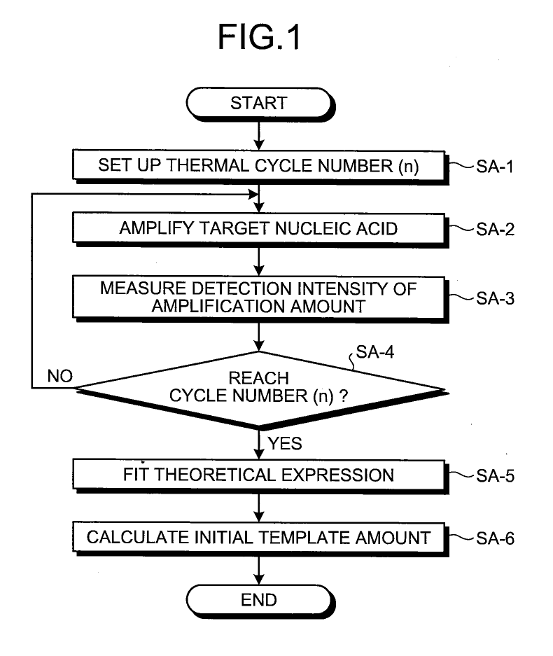

Hereinafter, an overview of the present embodiment will be explained with reference to FIG. 1, and then a configuration and processing of the present embodiment will be explained in detail. FIG. 1 is a flowchart for explaining an overview of the present embodiment.

As shown in FIG. 1, according to the present embodiment, first, the thermal cycle number (n) of a nucleic acid amplification reaction is set up (step SA-1). That is, the thermal cycle number (n) is set up for repeatedly performing the nucleic acid amplification reaction.

According to the present embodiment, the nucleic acid amplification reaction of the test sample is performed to amplify the target nucleic acid (step SA-2). Here, the test sample may contain an internal standard. The “internal standard” is a static standard that is added to the test sample in a certain amount and is not affected by the nucleic acid amplification reaction. For example, the internal standard may be a fluorescent dye added to the test sample in a certain amount. When using a fluorescent dye as an internal standard, a wavelength of the fluorescent dye should be different from that of fluorescence for measuring the detection intensity of the amplification amount of the target nucleic acid. The reason for using this internal standard is for the correction of the detection intensity (detection luminance or the like) between the thermal cycles in a same test sample, or for the correction of the detection intensity (detection luminance or the like) between test samples (between wells). That is, during the measurement of a same test sample, it is ideally required that there should be no fluctuation in the characteristics of excitation or detection, or no deactivation of the fluorescent dye or the like, between the thermal cycles. However, in practice, since there occurs fluctuation in the detection intensity that is not related to the increase of the target nucleic acid, the internal standard in a constant amount is added to the test sample to carry out internal standard correction by normalizing the detection intensity for each thermal cycle so that the detection intensity of the internal standard becomes 1. Furthermore, during the measurement of a plurality of test samples, it is ideally required that there should be no variability in the dispense amount between wells, or no variability in the characteristics of excitation or detection of the measuring apparatus. However, in practice, since there occurs unevenness in the dispense amount or unevenness in the excitation or detection, the internal standard is added to each well at the same concentration to carry out internal standard correction by normalizing the detection intensity (detection luminance or the like) for each of the wells so that the detection intensity of the internal standard becomes 1.

According to the present embodiment, a detection intensity of the amplification amount of the target nucleic acid is measured corresponding to the thermal cycle number (step SA-3). Here, the “amplification amount” means the total amount of the target nucleic acid amplified from thermal cycle number 1 to the thermal cycle number when measuring. Also, the “detection intensity of the amplification amount” is the detection intensity that is obtained by an arbitrary label and reflects the amplification amount of the target nucleic acid. For example, the “detection intensity of the amplification amount” is a signal intensity detected from an intercalator that emits the signal in the presence of a double-strand DNA, or a signal, intensity detected from a reporter molecule that emits the signal according to an extension reaction during the amplification reaction. According to the present embodiment, since the measured detection intensity of the amplification amount is directly used to fit a theoretical expression, the “measured detection intensity of the amplification amount” is not perfectly coincident with the amount equivalent to the amplification amount of the target nucleic acid, in a strict sense. The “measured detection intensity of the amplification amount” is an unprocessed measurement data, that is, a raw data including the fluctuation in the detection intensity, a background value, and the like that is not related to the nucleic acid amplification reaction. In this step SA-3, the detection intensity of the internal standard (for example, the luminance value of the fluorescent dye added as an internal standard) may be measured simultaneously with the measurement of the detection intensity of the amplification amount.

According to the present embodiment, it is determined whether current thermal cycle number has reached the set thermal cycle number (n) (step SA-4), and when the current thermal cycle number has not reached the set thermal cycle number (n) (step SA-4, No), the process is returned to the step SA-2. On the other hand, when the current thermal cycle number has reached the set thermal cycle number (n) (step SA-4, Yes), the above-mentioned processes are terminated.

According to the present embodiment, a theoretical expression is fitted to the detection intensity of the amplification amount, measured in each thermal cycle (step SA-5). The theoretical expression includes an environmental coefficient as an exponent parameter, a parameter of an initial template amount in the test sample, and terms for the internal standard correction and baseline correction of the measured detection intensity of the amplification amount. Further, the theoretical expression includes at least one parameter among a saturation amount upon the target nucleic acid amplification, a reaction acceleration coefficient, and a reaction inhibition coefficient. That is, according to the present embodiment, the parameters of the theoretical expression are determined in this step SA-5 so as to fit the theoretical expression to the detection intensity of the amplification amount measured in each thermal cycle.

Here, the terms for the internal standard correction and the baseline correction in the theoretical expression may include a term that performs the internal standard correction, and a term that performs the baseline correction. The former term is a term that performs the internal standard correction by dividing the measured detection intensity of the amplification amount by the measured detection intensity of the internal standard for each thermal cycle number. The latter term is a term that performs the baseline correction by subtracting from the term that performs the internal standard correction, a value obtained by dividing a background value of the detection intensity of the amplification amount by a background value of the detection intensity of the internal standard. Here, the “background value” is a value of a background that is not related to the amount of the target nucleic acid. For example, the background value is the detection intensity measured before an amplification curve occurs in the nucleic acid amplification reaction, or the detection intensity measured at thermal cycle number 0. In addition, the background value is not limited to a constant value, but may be a value that fluctuates over the thermal cycles.

In the theoretical expression, an amplification efficiency of the nucleic acid amplification reaction may be defined by the environmental coefficient and at least one parameter among the saturation amount upon the target nucleic acid amplification, the reaction acceleration coefficient, and the reaction inhibition coefficient. The theoretical expression may express that the amplification amount of the target nucleic acid derived using the terms for the internal standard correction and the baseline correction, is equal to a number obtained by subtracting the initial template amount from the product of the initial template amount and a sequence of the amplification efficiencies to each of which 1 is added for each thermal cycle number. According to the present embodiment, the theoretical expression may be fitted using the least square method.

According to the present embodiment, the initial template amount is calculated from the theoretical expression fitted to the detection intensity of the amplification amount (step SA-6). For example, according to the present embodiment, the initial template amount is calculated based on the theoretical expression in which the parameters have been determined by fitting.

An overview of the present embodiment has been explained hereinbefore. According to the present embodiment, the initial template amount can be precisely determined using a theoretical expression that allows to be fitted to the measured detection intensity of the amplification amount in the form of raw data and is capable of precisely reflecting the actual amplification efficiency.

Configuration of Target Nucleic Acid Measuring Apparatus

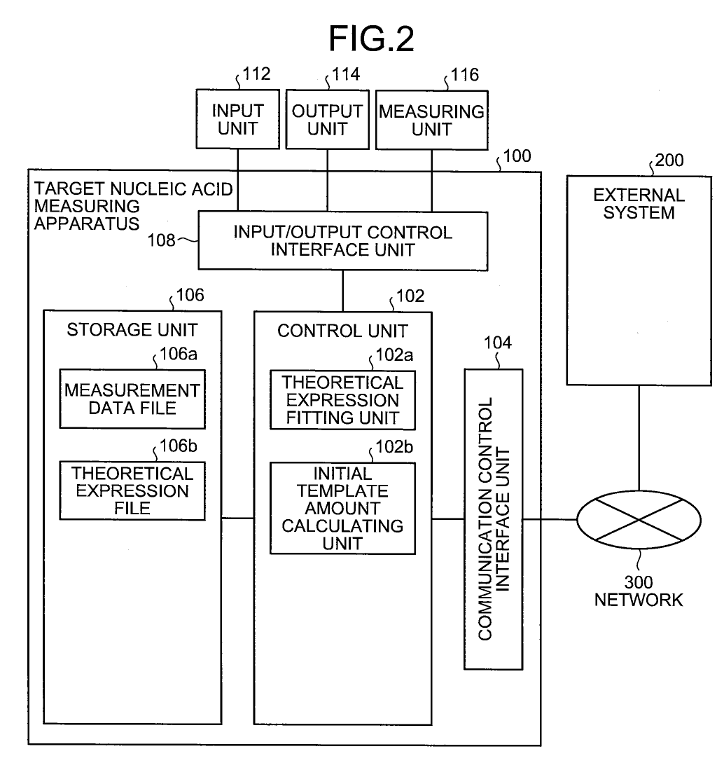

Next, a configuration of a target nucleic acid measuring apparatus according to the present embodiment will be explained below with reference to FIG. 2. FIG. 2 is a block diagram showing an example of a configuration of a target nucleic acid measuring apparatus 100 according to an embodiment to which the present invention is applied, and schematically depicts only a part related to the present invention in the configuration.

In FIG. 2, the target nucleic acid measuring apparatus 100 schematically includes a control unit 102, a communication control interface unit 104, an input/output control interface unit 108, and a storage unit 106. The control unit 102 is a CPU and the like that integrally controls the entire operation of the target nucleic acid measuring apparatus 100. The input/output control interface unit 108 is an interface connected to an input unit 112, an output unit 114, and a measuring unit 116. The storage unit 106 is a device that stores various databases, tables or the like. These components of the target nucleic acid measuring apparatus 100 are communicably connected through an arbitrary communication path.

The various databases or files (a measurement data file 106a and a theoretical expression file 106b) stored in the storage unit 106 are storage means such as a fixed disk device. For example, the storage unit 106 stores various programs, tables, files, databases and the like which are used in various processes.

Of these constituent elements of the storage unit 106, the measurement data file 106a stores a detection intensity as a raw data (unprocessed measurement data) reflecting the amplification amount of the target nucleic acid, which are measured at each thermal cycle number, corresponding to the thermal cycle number in the nucleic acid amplification reaction. For example, the nucleic acid amplification reaction is PCR. The measurement data file 106a may store a detection intensity of an internal standard which is measured simultaneously with the measurement of the detection intensity of the amplification amount for each thermal cycle number.

The theoretical expression file 106b stores a theoretical expression. The theoretical expression stored in the theoretical expression file 106b includes an environmental coefficient K as an exponent parameter, a parameter of an initial template amount N₀, and terms for internal standard correction and baseline correction of the detection intensity of the amplification amount. Further, the theoretical expression includes at least one parameter among a saturation amount Nₘₐₓ upon the target nucleic acid amplification, a reaction acceleration coefficient ρ, and a reaction inhibition coefficient μ.

Here, the terms for the internal standard correction and the baseline correction in the theoretical expression stored in the theoretical expression file 106b may include a term that performs the internal standard correction, and a term that performs the baseline correction. The former term is a term that performs the internal standard correction by dividing the measured detection intensity of the amplification amount by the measured detection intensity of the internal standard for each thermal cycle number. The latter term is a term that performs the baseline correction by subtracting from the term that performs the internal standard correction, a value (baseline) obtained by dividing a background value of the detection intensity of the amplification amount by a background value of the detection intensity of the internal standard. In the theoretical expression stored in the theoretical expression file 106b, an amplification efficiency of the nucleic acid amplification reaction may be defined by the environmental coefficient K and at least one parameter among the saturation amount Nₘₐₓ upon the target nucleic acid amplification, the reaction acceleration coefficient ρ, and the reaction inhibition coefficient μ.

Further, the theoretical expression may express that the amplification amount of the target nucleic acid derived using the terms for the internal standard correction and the baseline correction, is equal to a number obtained by subtracting the initial template amount N₀ from the product of the initial template amount N₀ and a sequence of the amplification efficiencies to each of which 1 is added for each thermal cycle number. More specifically, this amplification amount of the target nucleic acid is derived by dividing the detection intensity of the amplification amount corrected by the terms for the internal standard correction and the baseline correction, by S₁ that denotes the detection intensity per unit of the target nucleic acid (hereinafter, referred to as “detection intensity per unit S₁”). That is, the detection intensity per unit S₁ is a coefficient for conversion of the detection intensity corrected (by the internal standard correction and the baseline correction) into a unit of target nucleic acid. In other words, when multiplying 1/S₁ by the corrected detection intensity of the amplification amount, the quantity expressed in the unit of the detection intensity of the amplification amount is converted to a quantity expressed in the unit of the target nucleic acid. For example, S₁ is the detection intensity (for example, a fluorescence intensity, luminance, or signal measurement value) per unit quantity (copy number, mass, amount of substance, concentration, or the like) representing an amount of the target nucleic acid (template amount).

For example, the theoretical expression stored in the theoretical expression file 106b is represented by the following expression.

$${N}_{0} [ {\prod }_{j = 1}^{n} \{ 1 + {ρ ( \frac{{N}_{max} - {N}_{j - 1}}{{N}_{max} + μ {N}_{j - 1}} )}^{K} \} - 1 ] = \frac{{I}_{00}}{{S}_{1}} ( \frac{{I}_{1 n}}{{I}_{0 n}} - \frac{{I}_{10}}{{I}_{00}} )$$

(Here, N₀ is the initial template amount; j is the thermal cycle number; Nⱼ is the amplification amount of the target nucleic acid at the thermal cycle number j; Nₘₐₓ is the saturation amount upon the target nucleic acid amplification; ρ is the reaction acceleration coefficient; μ is the reaction inhibition coefficient; K is the environmental coefficient; S₁ is the detection intensity per unit; I₁ₙ, is the detection intensity of the amplification amount at the thermal cycle number n; and I₀ₙ is the detection intensity of the internal standard at the thermal cycle number n. The right-hand side of this theoretical expression represents the amplification amount of the target nucleic acid derived using the terms for the internal standard correction and the baseline correction.)

The theoretical expression file 106b may store a value of the parameter in the theoretical expression. For example, the theoretical expression file 106b may store, as a fixed value, at least one value of the detection intensity per unit S₁, the reaction acceleration coefficient ρ, the reaction inhibition coefficient μ, the environmental coefficient K, and the saturation amount Nₘₐₓ upon the target nucleic acid amplification.

In FIG. 2, the input/output control interface unit 108 controls the input unit 112, the output unit 114, and the measuring unit 116. As the output unit 114, not only a monitor (including a household-use television) but also a speaker may be used (hereinafter, the output unit 114 is sometimes described as a monitor). As the input unit 112, a keyboard, a mouse device, a microphone or the like may be used.

The measuring unit 116 measures the detection intensity of the amplification amount or the detection intensity of the internal standard at each thermal cycle number, corresponding to the thermal cycle number in the nucleic acid amplification reaction. As an example, the measuring unit 116 is constituted as a measuring means in a real-time PCR apparatus or the like. In addition, the detection intensity of the amplification amount or the detection intensity of the internal standard for each thermal cycle number as measured by the measuring unit 116, is stored in the measurement data file 106a in the form of raw data (unprocessed measurement data) under the control of the control unit 102.

In FIG. 2, the control unit 102 has an internal memory to store a control program such as an OS (Operating System), a program that defines various procedures, and required data. The control unit 102 performs information processing to execute various processes by these programs or the like. The control unit 102 functionally conceptually includes a theoretical expression fitting unit 102a, and an initial template amount calculating unit 102b.

The theoretical expression fitting unit 102a fits the theoretical expression stored in the theoretical expression file 106b, to the detection intensity of the amplification amount or the detection intensity of the internal standard for each thermal cycle number stored in the measurement data file 106a. That is, the theoretical expression fitting unit 102a determines values of the parameters in the theoretical expression so that the theoretical expression has the best fit to the detection intensity of the amplification amount or the detection intensity of the internal standard, which is measured at each thermal cycle number.

Here, the theoretical expression fitting unit 102a may fit the theoretical expression using the least square method. The theoretical expression fitting unit 102a may read out the value of the parameters stored in the theoretical expression file 106b, such as the detection intensity per unit S₁, the reaction acceleration coefficient ρ, the reaction inhibition coefficient μ, the environmental coefficient K, or the saturation amount upon the target nucleic acid amplification Nₘₐₓ. And then, the theoretical expression fitting unit 102a may use a predetermined value such as the read-out value of at least one of these parameters as a fixed value when fitting the theoretical expression. The theoretical expression fitting unit 102a may perform the fitting of the theoretical expression by setting the maximum value of the amplification amount derived using the terms for the internal standard correction and the baseline correction, as the saturation amount upon the target nucleic acid amplification Nₘₐₓ. For example, the maximum value of the values calculated by the right-hand side of the aforementioned theoretical expression, may be set as the saturation amount upon the target nucleic acid amplification Nₘₐₓ. The theoretical expression fitting unit 102a may fit the theoretical expression to the detection intensity of the amplification amount measured only in a region where the increasing rate Δₙ represented by the following expression is from 0 to 1 in order to fix the parameters such as the reaction acceleration coefficient ρ or the reaction inhibition coefficient μ. That is, the theoretical expression fitting unit 102a may fit the theoretical expression only to the detection intensity measured at the thermal cycle number of which the increasing rate Δₙ is from 0 to 1. And then, the theoretical expression fitting unit 102a may store, as a fixed value, a value of the parameters such as the reaction acceleration coefficient ρ or the reaction inhibition coefficient μ determined from the fitted theoretical expression, in the theoretical expression file 106b.

$${Δ}_{n} = \frac{{N}_{n}}{{N}_{n - 1}} - 1$$

(Here, Δₙ is the increasing rate; and Nₙ is the amplification amount of the target nucleic acid at thermal cycle number n.)

The initial template amount calculating unit 102b calculates the initial template amount from the theoretical expression fitted by the theoretical expression fitting unit 102a. For example, the initial template amount calculating unit 102b calculates the initial template amount N₀ based on the values of the parameters in the theoretical expression, which are the results of the fitting performed by the theoretical expression fitting unit 102a, to output the initial template amount N₀ to the output unit 114. The unit of the initial template amount depends on the detection intensity per unit S1 aforementioned, and examples thereof include the copy number (copy), mass (pg, ng), amount of substance (mol), concentration (mol/ml), and the like.

An example of the configuration of the target nucleic acid measuring apparatus 100 has been explained hereinbefore. The target nucleic acid measuring apparatus 100 may be communicably connected to a network 300 through a communication device such as a router and a wired or radio communication line such as a leased line. In this case, the communication control interface unit 104 is an interface connected to a communication device (not shown) such as a router connected to the communication line or the like, and performs communication control between the target nucleic acid measuring apparatus 100 and the network 300 (or a communication device such as a router). Namely, the communication control interface unit 104 has a function of performing data communication with another terminal through a communication line. In FIG. 2, the network 300 has a function of connecting the target nucleic acid measuring apparatus 100 and an external system 200 with each other. For example, the Internet is used as the network 300.

The target nucleic acid measuring apparatus 100 may be connected to the external system 200 which provides an external program making a computer serve as the target nucleic acid measuring apparatus, or an external database related to the measurement data or the parameters, through the network 300.

In FIG. 2, the external system 200 is mutually connected to the target nucleic acid measuring apparatus 100 through the network 300. And the external system 200 has a function of providing an external database related to the measurement data, the theoretical expressions, or values of the parameters, and an external program such as a target nucleic acid measuring program that makes an information processing device serve as the target nucleic acid measuring apparatus, to a user. The external system 200 may be designed to serve as a WEB server or an ASP server. The hardware configuration of the external system 200 may be constituted by an information processing device such as a commercially available workstation or personal computer and a peripheral device thereof. The functions of the external system 200 are realized by a CPU, a disk device, a memory device, an input unit, an output unit, a communication control device, and the like in the hardware configuration of the external system 200 and programs which control these devices.

Processing of Target Nucleic Acid Measuring Apparatus 100

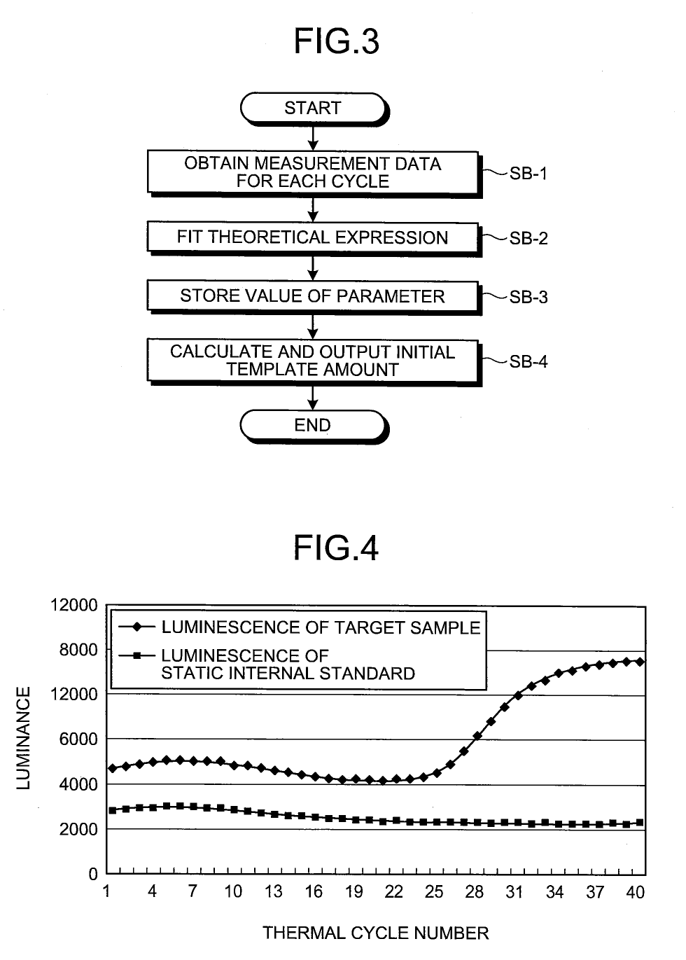

Next, an example of processing of the target nucleic acid measuring apparatus 100 according to the present embodiment constructed as described above will be explained below in detail with reference to FIG. 3. FIG. 3 is a flowchart showing an example of the processing of the target nucleic acid measuring apparatus 100 according to the present embodiment.

The measuring unit 116 of a real-time PCR measuring apparatus or the like measures a detection intensity of the amplification amount or a detection intensity (for example, a luminance value of the fluorescent dye) of the internal standard at each thermal cycle number, corresponding to the thermal cycle number in the nucleic acid amplification reaction. For example, the detection intensity of the amplification amount is a detection intensity such as a measurement value of a signal from an intercalator, a reporter molecule or the like. As shown in FIG. 3, the control unit 102 of the target nucleic acid measuring apparatus 100 obtains unprocessed measurement data (raw data) on the detection intensity of the amplification amount or the internal standard, which is measured at each thermal cycle number through the measuring unit 116 (step SB-1). The measurement data is stored in the measurement data file 106a.

The theoretical expression fitting unit 102a fits the theoretical expression stored in the theoretical expression file 106b, to the detection intensity of the amplification amount or the internal standard for each thermal cycle number stored in the measurement data file 106a (step SB-2). That is, the theoretical expression fitting unit 102a adjusts and determines values of the parameters in the theoretical expression using the least square method so that the theoretical expression has the best fit to the detection intensity of the amplification amount for each thermal cycle number. In the theoretical expression used by the theoretical expression fitting unit 102a, the terms for the internal standard correction and the baseline correction may include a term that performs the internal standard correction, and a term that performs the baseline correction. The former term is a term that performs the internal standard correction by dividing the detection intensity of the amplification amount by the detection intensity of the internal standard for each thermal cycle number. The latter term is a term that performs the baseline correction by subtracting from the term that performs the internal standard correction, a value (baseline) obtained by dividing a background value of the detection intensity of the amplification amount by a background value of the detection intensity of the internal standard. In this theoretical expression, an amplification efficiency of the nucleic acid amplification reaction may be defined by not only the environmental coefficient K, but also a parameter such as the saturation amount Nₘₐₓ upon the target nucleic acid amplification, the reaction acceleration coefficient ρ, or the reaction inhibition coefficient μ. As an example, the theoretical expression is a expression in which the amplification amount of the target nucleic acid derived using the terms for the internal standard correction and the baseline correction, is expressed equal to a number obtained by subtracting the initial template amount N₀ from the product of the initial template amount N₀ and a sequence of the amplification efficiencies to each of which 1 is added for each thermal cycle number. That is, the theoretical expression may express that the amplification amount of the target nucleic acid derived using the two terms is equal to a number obtained by subtracting the initial template amount N₀ from the product of the initial template amount N₀ and a sequence (Eₖ+1) (k=1, 2, . . . n), wherein Eₖ denotes the amplification efficiency at thermal cycle number k. Exemplarily, the theoretical expression is represented by the following expression.

$${N}_{0} [ {\prod }_{j = 1}^{n} \{ 1 + {ρ ( \frac{{N}_{max} - {N}_{j - 1}}{{N}_{max} + μ {N}_{j - 1}} )}^{K} \} - 1 ] = \frac{{I}_{00}}{{S}_{1}} ( \frac{{I}_{1 n}}{{I}_{0 n}} - \frac{{I}_{10}}{{I}_{00}} )$$

(Here, N₀ is the initial template amount; j is the thermal cycle number; Nⱼ is the amplification amount of the target nucleic acid at the thermal cycle number j; Nₘₐₓ is the saturation amount upon the target nucleic acid amplification; ρ is the reaction acceleration coefficient; μ is the reaction inhibition coefficient; K is the environmental coefficient; S₁ is the detection intensity per unit; I₁ₙ is the detection intensity of the amplification amount at the thermal cycle number n; and I₀ₙ is the detection intensity of the internal standard at the thermal cycle number n.)

The theoretical expression fitting unit 102a stores each value of the parameters of the fitted theoretical expression, in the theoretical expression file 106b (step SB-3). For example, the theoretical expression fitting unit 102a stores, as a result of the fitting, those values of the parameters optimized using the least square method so that the theoretical expression has the best fit to the detection intensity of the amplification amount, in the theoretical expression file 106b.

The initial template amount calculating unit 102b calculates the initial template amount N₀ based on the values of the parameters of the theoretical expression, stored in the theoretical expression file 106b, to output the initial template amount N₀ to the output unit 114 through the input/output control interface unit 108 (step SB-4).

An example of the processing of the target nucleic acid measuring apparatus 100 is described hereinbefore. According to the present embodiment, the theoretical expression is fitted to the detection intensity of the amplification amount measured at each thermal cycle number in the nucleic acid amplification reaction, and the initial template amount N₀ is calculated from the fitted theoretical expression. The theoretical expression includes an environmental coefficient K as an exponent parameter, a parameter of an initial template amount N₀, and terms for the internal standard correction and the baseline correction, and further includes at least one parameter among a saturation amount Nₘₐₓ, upon the target nucleic acid amplification, a reaction acceleration coefficient ρ, and a reaction inhibition coefficient μ. Therefore, according to the present embodiment, the initial template amount can be precisely determined using a theoretical expression that allows to be fitted to the measured detection intensity itself of the amplification amount in the form of raw data and is capable of precisely reflecting the actual amplification efficiency.

According to the present embodiment, the terms for the internal standard correction and the baseline correction in the theoretical expression includes a term that performs the internal standard correction, and a term that performs the baseline correction. The former term is a term that performs the internal standard correction by dividing the measured detection intensity of the amplification amount by the measured detection intensity of the internal standard for each thermal cycle number. The latter term is a term that performs the baseline correction by subtracting from the term that performs the internal standard correction, a value (baseline) obtained by dividing the background value of the detection intensity of the amplification amount by the background value of the detection intensity of the internal standard. Therefore, according to the present embodiment, the initial template amount can be more precisely determined using the theoretical expression in which the internal standard correction and the baseline correction can be performed to allow to be fitted to raw data without preprocessing the measured detection intensity of the amplification amount.

According to the present embodiment, an amplification efficiency of the nucleic acid amplification reaction is defined in the theoretical expression by the environmental coefficient K and at least one parameter among the saturation amount Nₘₐₓ upon the target nucleic acid amplification, the reaction acceleration coefficient ρ, and the reaction inhibition coefficient μ. Therefore, according to the present embodiment, the initial template amount can be more precisely determined using the theoretical expression that is capable of more precisely reflecting the actual amplification efficiency.

According to the present embodiment, the theoretical expression expresses that the amplification amount of the target nucleic acid derived using the terms for the internal standard correction and the baseline correction, is equal to a number obtained by subtracting the initial template amount N₀ from the product of the initial template amount N₀ and a sequence of the amplification efficiencies to each of which 1 is added for each thermal cycle number. Therefore, according to the present embodiment, the initial template amount can be more precisely determined using the theoretical expression that allows to be fitted to the measured detection intensity of the amplification amount in the form of raw data and is capable of more precisely reflecting the actual amplification efficiency.

According to the present embodiment, the theoretical expression is represented by the following expression, thereby enabling to determine the initial template amount more precisely using the theoretical expression that is capable of more precisely reflecting the actual amplification efficiency.

$${N}_{0} [ {\prod }_{j = 1}^{n} \{ 1 + {ρ ( \frac{{N}_{max} - {N}_{j - 1}}{{N}_{max} + μ {N}_{j - 1}} )}^{K} \} - 1 ] = \frac{{I}_{00}}{{S}_{1}} ( \frac{{I}_{1 n}}{{I}_{0 n}} - \frac{{I}_{10}}{{I}_{00}} )$$

(Here, N₀ is the initial template amount; j is the thermal cycle number; Nⱼ is the amplification amount of the target nucleic acid at the thermal cycle number j; Nₘₐₓ is the saturation amount upon the target nucleic acid amplification; μ is the reaction acceleration coefficient; μ is the reaction inhibition coefficient; K is the environmental coefficient; S₁ is the detection intensity per unit; I₁ₙ, is the detection intensity of the amplification amount at the thermal cycle number n; and I₀ₙ is the detection intensity of the internal standard at the thermal cycle number n.)

According to the present embodiment, the theoretical expression is fitted using the least square method, thereby enabling to perform the fitting and determine the initial template amount, more precisely.

### Example 1

Example 1 according to the present embodiment will be described hereinafter with reference with FIGS. 4 to 6.

Theoretical Expression

First, a theoretical expression to be used in Example 1 will be described bellow.

In the real-time PCR system according to this Example 1, the total intensity I₀ₙ in an image of the static internal standard and the total intensity I₁ₙ in an image of a sample (target nucleic acid), which are obtained using two-types of filters at thermal cycle number n, are represented by the following expressions.

I₀ₙ=φₙ(S₀N₀ₙ+I_{0B} (1)

I₁ₙ=φₙ(S₁N₁ₙ+I_{1B} (2)

(Here, N₀ₙ and N₁ₙ respectively denote the number of static internal standards and DNA copy number of the sample at thermal cycle number n; I_{0B} and I_{1B} respectively denote a background of the static internal standard and that of the sample; and φₙ is detection sensitivity profile resulting from reaction environment, and gives the following expression when n=0.)

φ₀=1 (3)

Here, the parameters used in the expressions (1) and (2) will be explained in more detail. When the wavelengths are completely separated by the filters, the two initial total intensities I_{R} in the vessel image of light-emitting reference using the two types of the filters, are given by the following expressions.

I_{R0}=m₀S₀ (1′)

I_{R1}=m₁S₁ (2′)

(Here, m₀ and m₁ respectively denote the number of molecules of the static internal standard (fluorescent dye that is not affected by the nucleic acid amplification reaction) and the number of molecules of the sample; and S₀ and S₁ respectively denote a detection sensitivity (a detection intensity per fluorescent molecule) of the static internal standard and that of the sample.)

The following expressions are given by the expressions (1′) and (2′).

$$\begin{matrix}{S}_{0} = \frac{{I}_{R 0}}{{m}_{0}} & ( {1}^{″} ) \\ {S}_{1} = \frac{{I}_{R 1}}{{m}_{1}} & ( {2}^{″} )\end{matrix}$$

The detection sensitivities S₀ and S₁ determined by the expressions (1″) and (2″) are critical quantities that serve as the coefficients when the quantity of fluorescent molecules is estimated from the luminance. It is required to verify beforehand the pixel density range where these detection sensitivities are maintained, that is, the dynamic range. To do this, it will be effective to change the number-of-molecules m (for example, by preparing a dilution series), and to calculate S(m) from the intensity obtained according to the change, to thereby produce an m-S curve. It is also important to know the pixel sensitivity distribution of the sensor, and if this sensitivity is not uniformly distributed, it is necessary to perform the sensitivity correction of the entire image. In this instance, it is assumed that all the pixel densities are within the dynamic range, and the pixel sensitivities are uniformly distributed.

Next, the solution needed in the PCR is composed of an appropriate buffer, two sets of complementary oligonucleotide primers, an excess amount of four nucleotide triphosphates, a DNA polymerase, an unknown amount of the target nucleic acid molecules, and the like. For such a composition, the amount of the PCR product, Nₙ, in the PCR reaction is represented by the following expression.

$${N}_{n} = {N}_{n - 1} ( 1 + ρ \frac{{N}_{max} - {N}_{n - 1}}{{N}_{max} + μ {N}_{n - 1}} ) p ( {N}_{zn - 1} , {A}_{zn - 1} , {T}_{x} , {L}_{t} , {L}_{p} ) \qquad ( 4 )$$

(Here, ρ is an amplification coefficient, and is ideally 1, but in practice, the value becomes from 0 to 1 depending on the conditions of the system (experimental conditions) (when ρ=0, no amplification); μ is an inhibition coefficient, and is ideally 0, but under the influence of an amplification inhibitory substance such as pyrophosphoric acid, the value becomes from 0 to ∞ (when μ=∞, no amplification); and p(N_{zn-1}, A_{zn-1}, Tₓ, Lₜ, Lₚ) is a performance characteristic function for the polymerases, and the value thereof depends on the number of the polymerases at thermal cycle number n−1 in the PCR, N_{zn-1}; specific activity of the polymerase at the thermal cycle number n−1, A_{zn-1}; the extension time, Tₓ (seconds); the base length of the template, Lₜ; and the base length of the primer, Lₚ.)

Since P(N_{zn1i}, A_{zn-1}, Tₓ, Lₜ, Lₚ)=1 when the polymerases function perfectly, the expression (4) becomes the following expression.

$${N}_{n} = {N}_{n - 1} ( 1 + ρ \frac{{N}_{max} - {N}_{n - 1}}{{N}_{max} + μ {N}_{n - 1}} ) \qquad ( 5 )$$

However, under the practical conditions, the polymerase is not believed to be functioning perfectly. Also, since a specific expression of the performance characteristic function for the polymerase, p(N_{zn-1}, A_{zn-1}, Tₓ, Lₜ, Lₚ), is not clearly known, a variable K is introduced in place of this function to change the expression (5) to the following expression in Example 1.

$${N}_{n} = {N}_{n - 1} \{ 1 + {ρ ( \frac{{N}_{max} - {N}_{n - 1}}{{N}_{max} + μ {N}_{n - 1}} )}^{K} \} \qquad ( 6 )$$

Here, Nₘₐₓ in the expressions (4), (5) and (6) denotes a saturation amount of DNA (the amount of DNA saturated when thermal cycles are sufficiently carried out in the PCR), and is represented by the following expression.

Nₘₐₓ=Nₚ₀+N₀ (7)

(Here, Nₚ₀ is the number of the initial primers; and N₀ is the number of the template DNAs.)

According to the expression (6), the number of DNAs at thermal cycle number n in the PCR is represented by the following expression.

$${N}_{n} = {N}_{0} {\prod }_{j = 1}^{n} \{ 1 + {ρ ( \frac{{N}_{max} - {N}_{j - 1}}{{N}_{max} + μ {N}_{j - 1}} )}^{K} \} \qquad ( 8 )$$

Further, according to the expression (6), a DNA increasing rate (amplification efficiency) at thermal cycle number n in the PCR, Δₙ, is represented by the following expression.

$${Δ}_{n} = \frac{{N}_{n}}{{N}_{n - 1}} - 1 = {ρ ( \frac{{N}_{max} - {N}_{n - 1}}{{N}_{max} + μ {N}_{n - 1}} )}^{K} \qquad ( 9 )$$

As is obvious from the expression (9), the relationship between the increasing rate Δₙ and the amplification coefficient ρ is theoretically represented by the following relational expression.

0≦Δₙ≦ρ≦1 (10)

The expressions (11) and (12) are obtained following from the expressions (1) and (2), respectively.

$$\begin{matrix}{N}_{0 n} = \frac{{I}_{0 n}}{{ϕ}_{n} {S}_{0}} - \frac{{I}_{0 B}}{{S}_{0}} & ( 11 ) \\ {N}_{1 n} = \frac{{I}_{1 n}}{{ϕ}_{n} {S}_{1}} - \frac{{I}_{1 B}}{{S}_{1}} & ( 12 )\end{matrix}$$

Since the static internal standard is not amplified during PCR, the following relational expression is held.

N₀ₙ=N₀₀ (13)

The following expression is given by the expressions (3) and (11).

$${N}_{00} = \frac{{I}_{00}}{{S}_{0}} - \frac{{I}_{0 B}}{{S}_{0}} \qquad ( 14 )$$

The following expression is given by the expressions (11) and (13)

$${N}_{00} = \frac{{I}_{0 n}}{{ϕ}_{n} {S}_{0}} - \frac{{I}_{0 B}}{{S}_{0}} \qquad ( 15 )$$

The following expression is given by the expressions (14) and (15).

$$\frac{1}{{ϕ}_{n}} = \frac{{I}_{00}}{{I}_{0 n}} \qquad ( 16 )$$

The following expression is given by the expressions (3) and (12).

$${N}_{1 n} - {N}_{10} = \frac{{I}_{1 n}}{{ϕ}_{n} {S}_{1}} - \frac{{I}_{10}}{{S}_{1}} \qquad ( 17 )$$

By substituting the expression (16) into the expression (17) and simplifying it, the following expression is given.

$${N}_{1 n} - {N}_{10} = \frac{{I}_{00}}{{S}_{1}} ( \frac{{I}_{1 n}}{{I}_{0 n}} - \frac{{I}_{10}}{{I}_{00}} ) \qquad ( 18 )$$

By substituting the expression (8) into the expression (18), the following expression is given.

$${N}_{10} [ {\prod }_{j = 1}^{n} \{ 1 + {ρ ( \frac{{N}_{max} - {N}_{j - 1}}{{N}_{max} + μ {N}_{j - 1}} )}^{K} \} - 1 ] = \frac{{I}_{00}}{{S}_{1}} ( \frac{{I}_{1 n}}{{I}_{0 n}} - \frac{{I}_{10}}{{I}_{00}} ) \qquad ( 19 )$$

(Here, the right-hand side of the expression (19) is a known quantity because it is given by constants and measurements, and from a physical viewpoint, the value is obtained by subtracting the baseline (I₁₀/I₀₀) from the data treated with static internal correction.)

According to this Example 1, the least square fitting is performed based on the theoretical expression (19) shown above. Specifically, the coefficients, ρ, μ, K, N₀ and Nₘₐₓ on the left-hand side of the theoretical expression (19) are determined by performing least square fitting using a plurality of expressions corresponding to n's in which Iₙ can be detected with high precision. Then, the value of N₀ thus determined is taken as the estimated quantity of the template DNA (initial template amount).

Here, the difference between the theoretical expression of this Example 1 constituted as described above, and conventional theoretical expressions, will be explained.

For example, the expression disclosed in JP-A-8-66199 includes the amounts of the primer and the polymerase as aforementioned. Here, in the expression shown in JP-A-8-66199, when the concentration is re-written by the number of DNAs, and the expression is re-written using an inhibition coefficient, the following expression is obtained.

$${N}_{n} = {N}_{n - 1} {e}_{V} + Min [ \begin{matrix}{N}_{n - 1} {e}_{V} ρ {\{ 1 + \frac{( 1 + μ ) {N}_{n - 1}}{{N}_{p 0} + {N}_{0} - {N}_{n - 1}} \}}^{- 1} , \\ \frac{{N}_{zn - 1} {A}_{zn - 1} {T}_{x}}{{L}_{t} - {L}_{p}}\end{matrix} ] \qquad ( 20 )$$

(Here, Nₙ₋₁ and Nₙ are the numbers of DNA at the thermal cycle numbers n−1 and n in a PCR, respectively; eᵥ is the DNA's probability of survival; ρ is an amplification coefficient; μ is an inhibition coefficient, N₀ is the number of template DNAs; Nₚ₀ is the number of initial primers; N_{zn-1} and A_{zn-1} are respectively the number of polymerases and the specific activity of the polymerase at thermal cycle number n−1 in the PCR; Tₓ is the extension time (seconds); Lₜ is the base length of the template; Lₚ is the base length of the primer; and the saturation amount of DNA, Nₘₐₓ, is given by the following expression.)

Nₘₐₓ=Nₚ₀+N₀ (21)

When the polymerases are fully functioning and eᵥ=1, the following expression is given by the expression (21)

$${N}_{n} = {N}_{n - 1} ( 1 + ρ \frac{{N}_{max} - {N}_{n - 1}}{{N}_{max} + μ {N}_{n - 1}} ) \qquad ( 22 )$$

Therefore, the expression (22) can be represented by the following expression.

$${N}_{n} = {N}_{0} {\prod }_{j = 1}^{n} ( 1 + ρ \frac{{N}_{max} - {N}_{j - 1}}{{N}_{max} + μ {N}_{j - 1}} ) \qquad ( 23 )$$

Next, JP-A-8-66199 discloses that the expression (20) can be represented by the following expression when Nₙ depends on the dysfunction of the polymerases.

$${N}_{n} = {N}_{n - 1} {e}_{V} + \frac{{N}_{zn - 1} {A}_{zn - 1} {T}_{x}}{{L}_{t} - {L}_{p}} \qquad ( 24 )$$

As such, the second term in the expression (24) derived from JP-A-8-66199 does not include Nₙ₋₁, and is not understandable. There is no explanation on the initial number of polymerase, N_{z0}, and the initial specific activity, A_{z0}, and thus the calculation cannot be carried out. Furthermore, the expression (20) employs the smaller of the two argument values in the second term, and the both arguments can be used for fitting in practice. Since it is not known which argument should be employed for the fitting, the expression (20) cannot be utilized in the fitting. Essentially, it is believed that upon performing the fitting, the expression including these two arguments should be arranged into a single expression without arguments, and only when the expression are arranged into one without arguments, a practical system can be established.

Compared with the expression described in this JP-A-8-66199, the theoretical expression (19) used in this Example 1 is presented as an expression unified throughout the entire thermal cycles of the PCR and includes a term for correction, and therefore, the raw data can be utilized in the fitting.

Furthermore, by introducing one exponent parameter (environmental coefficient K) in place of the performance characteristic function for the polymerases which fluctuates with the thermal cycles of the PCR, various parameters can be omitted. Examples of the parameters that can be omitted include the number of polymerases, the specific activity of the polymerase, the extension time, the base length of the template, and the base length of the primer. By omitting these various parameters, fitting of the theoretical expression can be made easy.

In the explanation given above, it was described such that the exponent parameter (environmental coefficient K) is introduced in place of the performance characteristic function for the polymerase. However, parameters to be replaced with this exponent parameter (environmental coefficient K) are not limited to the parameters associated with the polymerase.

That is, in regard to the actual nucleic acid amplification reaction such as PCR and measurements thereof, the actual amplification efficiency based on the measured detection intensity of the amplification amount is influenced not only by the performance characteristics of the polymerase, but also by various known factors or unknown factors. In this Example 1, it was found that by introducing the exponent parameter (environmental coefficient K) into the amplification efficiency in the theoretical expression, those parameters based on such diverse factors can be replaced with the single parameter, and the calculated amplification efficiency can be highly coincident with the actual amplification efficiency.

As an example, those factors already known to exert influence on the environmental coefficient K include the following. That is, examples of the factors associated with the measuring apparatus include the factors resulting from the thermal cycler, such as the error relative to the set temperature, the rate of temperature increase, the rate of temperature decrease or the like as well as the factors resulting from the light measuring unit, such as the sensitivities (of CCD and the light source), the image density conversion treatment or the like. Examples of the factors associated with the application include the heat resistance of the enzymes (polymerase and the like), the difference in the amplification efficiency due to the primer sequence, the difference in the amplification efficiency due to the type of the enzymes (polymerase and the like), and the like. As such, the environmental coefficient K as an exponent parameter that is introduced into the amplification efficiency of the theoretical expression, can be a substitute for these parameters associated with such known factors and unknown factors, which are currently not known.

Internal Standard Correction and Baseline Correction

The terms for internal standard correction and baseline correction in right-hand side of the theoretical expression (19) according to the Example 1, will be explained with tangible experimental data in reference with FIGS. 4 to 6. That is, the theoretical expression (19) will be explained according to new analysis flow by measuring apparatus ABI7900 (trade name) manufactured by Applied Biosystems, Inc. FIG. 4 is a diagram showing raw data (unprocessed measurement data) regarding detection intensity (luminance) of the amplification amount of the target sample (target nucleic acid) and that of the static internal standard, which are obtained by the measuring apparatus ABI7900 (trade name).

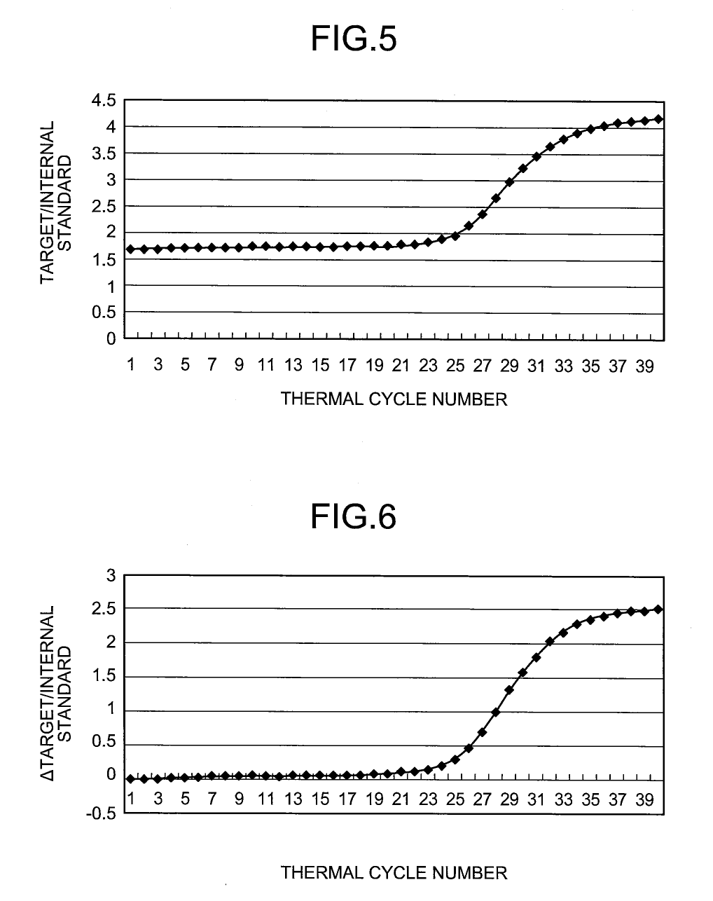

In the theoretical expression (19), when the parameters are set such that S₁=1, I₀₀=2784, and I₁₀=4663, the right-hand side of this theoretical expression is in the form of (I₁ₙ/I₀ₙ−1.675) multiplied by a proportional constant 2784. Furthermore, this 1.675 is called as a baseline. Here, FIG. 5 is a diagram showing the luminance ratio data (I₁ₙ/I₀ₙ) of the target sample and the internal standard, calculated from the raw data shown in FIG. 4.

As shown in FIG. 4, the internal standard correction can be performed by determining the luminance ratio of the target sample and the internal standard. Here, FIG. 6 is a diagram showing the data (I₁ₙ/I₀ₙ−1.675) obtained by subtracting the baseline 1.675 from the luminance ratio shown in FIG. 5.

As shown in FIG. 6, the baseline correction can be carried out by further subtracting the baseline 1.675. That is, when unprocessed measurement data are assigned into the right-hand side of the theoretical expression (19), the entirety of this right-hand side presents the measurement data obtained after performing the internal standard correction and the baseline correction. In this Example 1, when fitting is performed using the function on the left-hand side of this theoretical expression (19), values of the parameters, ρ, μ, K, Nₘₐₓ, and N₀, can be determined.

### Example 2

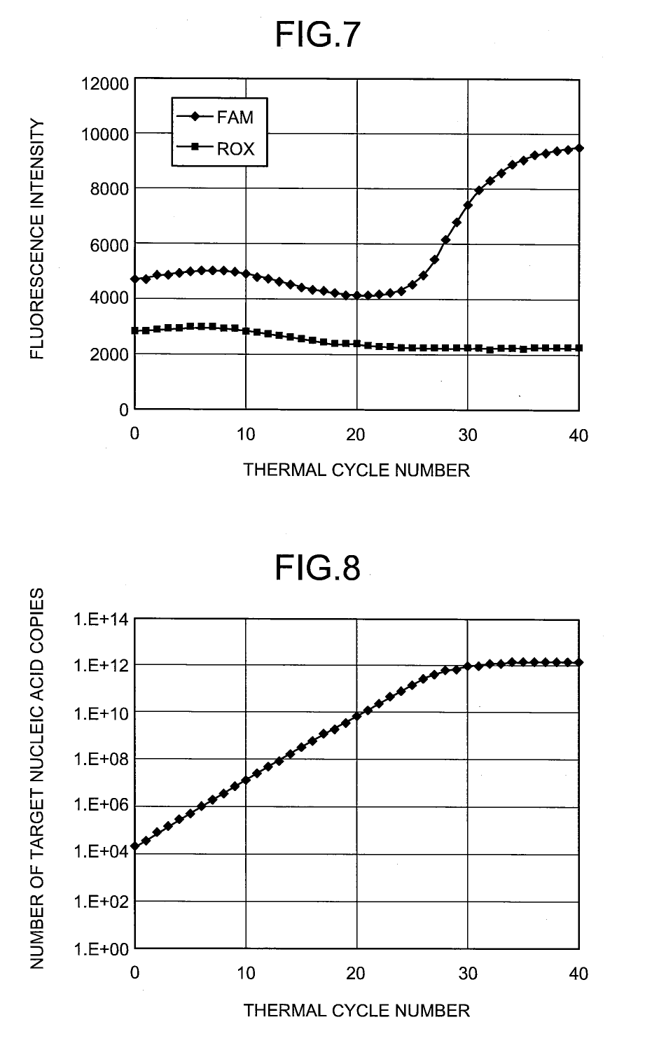

Example 2 according to the present embodiment will be described hereinafter with reference with FIGS. 7 to 11.

First, the measurement conditions in this Example 2 will be presented below. In this Example 2, the PCR reaction liquid was prepared as follows.

### <Composition of Reaction Liquid (Final Concentration (f.c.))>

2× Universal Master Mix (trade name) (manufactured by Applied Biosystems, Inc.): 1× dilution concentration

primers for human B2M: 0.3 μM each

TaqMan probe (trade name): 0.2 μM

plasmid DNA (plasmid in which a B2M gene is cloned into pCR2.1-TOPO vector (trade name)): 20000 copies

Total: 30 μl

Furthermore, the PCR conditions in Example 2 were as follows. As the real-time PCR apparatus, ABI Prism 7900HT (trade name) (manufactured by Applied Biosystems, Inc.) was used. The PCR cycle conditions were as follows: 60° C./1 minute→95° C./10 minutes→(95° C./15 seconds→60° C./1 minute)×40 cycles. Here, the fluorescence intensity of FAM (fluorescent dye for the detection of the number of DNA copies) and that of ROX (static internal standard) were measured during the period of 60° C. in each of the thermal cycles. FIG. 7 is a diagram showing the measurement result of FAM and ROX in the test sample, measured in the above measurement condition.

In FIG. 7, FAM plot reflects fluorescence intensity of the reporter molecule of TaqMan probe (trade name) and is data representing detection intensity of amplification amount of the target nucleic acid in the test sample. In FIG. 7, ROX plot reflects fluorescence intensity of the static internal standard in the test sample.

Since I₁₀=4663 and I₀₀=2784 were able to be determined by the raw data of FAN and ROX as shown in FIG. 7, the following theoretical expression was fitted for the measurement result of FIG. 7 (that is, I₁ₙ and I₀ₙ) as well as these values of I₁₀ and I₀₀.

$${N}_{0} [ {\prod }_{j = 1}^{n} \{ 1 + {ρ ( \frac{{N}_{max} - {N}_{j - 1}}{{N}_{max} + μ {N}_{j - 1}} )}^{K} \} - 1 ] = \frac{{I}_{00}}{{S}_{1}} ( \frac{{I}_{1 n}}{{I}_{0 n}} - \frac{{I}_{10}}{{I}_{00}} )$$

(Here, N₀ is the initial copy number of the target nucleic acid; Nₘₐₓ is the saturation amount upon the target nucleic acid amplification; ρ is the reaction acceleration coefficient; μ is the reaction inhibition coefficient; K is the environmental coefficient; Nₙ is the copy number of the target nucleic acid at thermal cycle number n; I₁ₙ is the fluorescence intensity of FAM at the thermal cycle number n; I₀ₙ is the fluorescence intensity of ROX at the thermal cycle number n; and S₁ is the detection sensitivity of FAM (the detection intensity per molecule of the fluorescence) and was set to 4.7×10⁻⁹ in Example 2.)

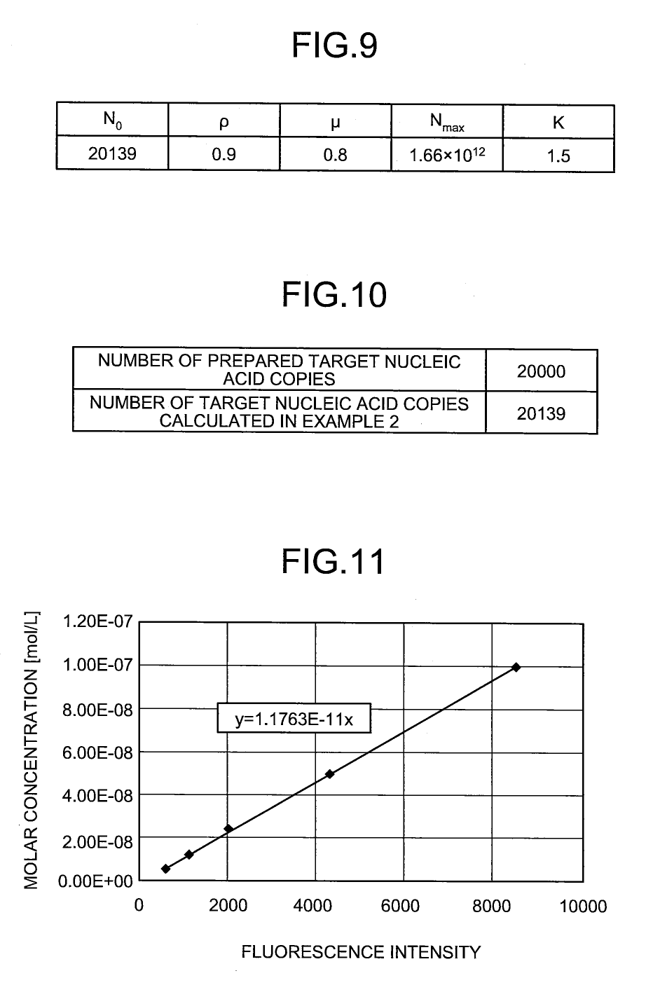

FIG. 8 is a diagram showing the result of the fitting. FIG. 9 is a diagram showing values of the parameters obtained by the fitting. As shown in FIG. 8, the values of the parameters on the left-hand side of the expression were determined so that the theoretical expression curve had the best fit for the corrected measurement data (right-hand side), and thus, as shown in FIG. 9, the values of the parameters could be determined by the fitting of this theoretical expression. Here, FIG. 10 is a diagram comparing the number of the prepared target nucleic acid copies, with the number of the target nucleic acid copies calculated in this Example 2.

As shown in FIG. 10, when the number of the prepared target nucleic acid copies was 20000, the number of the target nucleic acid copies calculated in this Example 2 was 20139. Thereby, it was confirmed that the initial copy number of the target nucleic acid is calculated with high precision by fitting the theoretical expression to the unprocessed measurement results (raw data).

Method for Calculation of S₁

In this Example 2, the value of S₁ was set to 4.7×10⁻⁹, but the method for the calculation of this S₁ will be described in the following.

First, a dilution series of FAM, which is a fluorescent dye for the detection of the number of DNA copies, was prepared such that the buffer composition would be the same as the PCR reaction liquid of the test sample, and the fluorescence intensity of FAM were measured under the same conditions as those for the PCR of the test sample. Here, FIG. 11 is a diagram showing the relationship between the molar concentration of FAM molecules and the fluorescence intensity.

As a result of measuring the FAM dilution series, “y=1.1763×10−11xx” is obtained as a relationship expression between the molar concentration of FAM molecules (y) and the fluorescence intensity (x), as shown in FIG. 11.

From this relationship expression, the fluorescence intensity when the molar concentration is 1 mol/l is 8.501×10¹⁰. Since the amount of solution in this case is 30 μl, the number of FAM molecules is (6.02×10²³)×(30×10⁻⁶)=1.81×10¹⁹. Therefore, the detection sensitivity of FAM (detection intensity per one fluorescent molecule), S₁, was calculated to be 8.501×10¹⁰/(1.81×10¹⁹)=4.7×10⁻⁹.

Thus, the explanation of this Example 2 will be ended. In the Example 2, the relationship expression of S₁ was derived under the premise that the relationship between the detection intensity (fluorescence intensity) of the amplification amount and the template amount is linearly expressed. However, the relationship is not intended to be limited thereto, but a non-linear relationship expression might be derived as far as it gives better approximation. Also, in the Example 2, S₁ is determined under the premise that one fluorescent molecule corresponds to one copy of DNA in order to calculate the initial number of DNA copies, but the present embodiment is not intended to be limited thereto. For example, a relationship expression may also be created by preparing a dilution series based on a unit such as copy number, mass, or amount of substance, according to the relationship between a label material such as an intercalator or a reporter molecule, and the template amount of nucleic acid, or according to the unit of the initial template amount that is wished to be determined. Then, the detection intensity per unit of the initial template amount that is wished to be determined, S₁, may be determined to utilize in the theoretical expression.

### Other Embodiments

The embodiments of the present invention have been described above. However, the present invention may be executed in not only the embodiments described above but also various different embodiments within the technical idea described in the scope of the invention.

For example, in the embodiment described above, the initial template amount (absolute quantitative value) was calculated by fitting using a theoretical expression to which S₁ had been introduced, but the present invention is not intended to be limited thereto. For example, without introducing S1 into the theoretical expression, an amount equivalent to the initial template amount (an initial template amount expressed in the unit of detection intensity) may be calculated, or the relationship (ratio, proportions, magnitude correlation, or the like) of the amounts equivalent to the initial template amounts between a plurality of test samples may also be calculated.

In the above embodiments, an example in which the target nucleic acid measuring apparatus 100 mainly performs the processes in a standalone mode is explained. However, as described in the embodiments, a process may be performed in response to a request from another terminal apparatus constituted by a housing different from that of the target nucleic acid measuring apparatus 100, and the process result may be returned to the client terminal.

Of each of the processes explained in the embodiments, all or some processes explained to be automatically performed may be manually performed. Alternatively, all or some processes explained to be manually performed may also be automatically performed by a known method. For example, a target nucleic acid measuring method as follows may be executed by a control unit.

(Note 1) A target nucleic acid measuring method of calculating an initial template amount in a test sample by fitting a theoretical expression to detection intensity of target nucleic acid for each thermal cycle number,

wherein the theoretical expression includes an environmental coefficient as an exponent parameter, a parameter of the initial template amount in the test sample, and terms for internal standard correction and baseline correction of the detection intensity of the amplification amount of the target nucleic acid, and includes at least one parameter among a saturation amount upon the target nucleic acid amplification, a reaction acceleration coefficient, and a reaction inhibition coefficient.

(Note 2) The target nucleic acid measuring method according to Note 1,

wherein the terms for the internal standard correction and the baseline correction in the theoretical expression, include:

a term that performs the internal standard correction by dividing the detection intensity of the amplification amount of the target nucleic acid by the detection intensity of an internal standard for each thermal cycle number; and

a term that performs the baseline correction by subtracting from the term that performs the internal standard correction, a value obtained by dividing the background value of the detection intensity of the amplification amount of the target nucleic acid by the background value of the detection intensity of the internal standard.

(Note 3) The target nucleic acid measuring method according to Note 1 or 2,

wherein an amplification efficiency of the nucleic acid amplification reaction is defined by the environmental coefficient and at least one parameter among the saturation amount upon the target nucleic acid amplification, the reaction acceleration coefficient, and the reaction inhibition coefficient, in the theoretical expression.

(Note 4) The target nucleic acid measuring method according to Note 3,

wherein the theoretical expression expresses that the amplification amount of the target nucleic acid derived using the terms for the internal standard correction and the baseline correction, is equal to a number obtained by subtracting the initial template amount from the product of the initial template amount and a sequence of the amplification efficiencies to each of which 1 is added for each thermal cycle number.

(Note 5) The target nucleic acid measuring method according to Note 4,

wherein the theoretical expression is represented by the following expression:

$${N}_{0} [ {\prod }_{j = 1}^{n} \{ 1 + {ρ ( \frac{{N}_{max} - {N}_{j - 1}}{{N}_{max} + μ {N}_{j - 1}} )}^{K} \} - 1 ] = \frac{{I}_{00}}{{S}_{1}} ( \frac{{I}_{1 n}}{{I}_{0 n}} - \frac{{I}_{10}}{{I}_{00}} )$$

(wherein N₀ is the initial template amount; j is the thermal cycle number; Nⱼ is the amplification amount of the target nucleic acid at the thermal cycle number j; Nₘₐₓ is the saturation amount upon the target nucleic acid amplification; ρ is the reaction acceleration coefficient; μ is the reaction inhibition coefficient; K is the environmental coefficient; S₁ is the detection intensity per unit of the target nucleic acid; I₁ₙ is the detection intensity of the amplification amount of the target nucleic acid at the thermal cycle number n; and I₀ₙ is the detection intensity of the internal standard at the thermal cycle number n).
(Note 6) The target nucleic acid measuring method according to any one of Notes 1 to 5,

wherein the theoretical expression is fitted using the least square method.

In addition, the procedures, the control procedures, the specific names, the information including parameters such as registered data or search condition, examples of screen image and the database configurations which are described in the literatures or the drawings may be arbitrarily changed unless otherwise noted.

With respect to the target nucleic acid measuring apparatus 100, the constituent elements shown in the drawings are functionally schematic. The constituent elements need not be always physically arranged as shown in the drawings.

For example, in the embodiment described above, the measuring unit 116 was constituted as one component included in the target nucleic acid measuring apparatus 100, but the present invention is not intended to be limited thereto. For example, the measuring unit 116 may be replaced with a measuring apparatus such as a real-time PCR apparatus, including a measuring unit that measures a detection intensity corresponding to each thermal cycle number in a nucleic acid amplification reaction. That is, the invention may be constituted as a target nucleic acid measuring system including a measuring apparatus with a measuring unit, and an information processing apparatus with a control unit and a storage unit. In this target nucleic acid measuring system, the measuring unit of the measuring apparatus has the same function as the measuring unit 116 according to the above embodiments, and the storage unit and the control unit of the information processing apparatus has the same function as the storage unit 106 and the control unit 102 of the target nucleic acid measuring apparatus 100 according to the above embodiments, and thus the system is constituted so as to have the similar effect. An example of the target nucleic acid measuring system is as follows.

A target nucleic acid measuring system comprising a measuring apparatus with a measuring unit that measures a detection intensity of an amplification amount of target nucleic acid at each thermal cycle number, and an information processing apparatus with a storage unit and a control unit,

wherein the storage unit includes:

an amplification amount detection intensity storage unit that stores the detection intensity of the amplification amount of the target nucleic acid, which is measured by the measuring apparatus corresponding to thermal cycle number; and

a theoretical expression storage unit that stores a theoretical expression including an environmental coefficient as an exponent parameter, a parameter of an initial template amount in the test sample, and terms for internal standard correction and baseline correction of the detection intensity of the amplification amount of the target nucleic acid, and including at least one parameter among a saturation amount upon the target nucleic acid amplification, a reaction acceleration coefficient, and a reaction inhibition coefficient, and

wherein the control unit includes:

a theoretical expression fitting unit that fits the theoretical expression to the detection intensity of the amplification amount of the target nucleic acid for each thermal cycle number stored in the storage unit; and

an initial template amount calculating unit that calculates the initial template amount from the theoretical expression fitted by the theoretical expression fitting unit.

In addition, all or some processing functions of the devices in the target nucleic acid measuring apparatus 100, in particular, processing functions performed by the control unit 102 may be realized by a central processing unit (CPU) and a program interpreted and executed by the CPU or may also be realized by hardware realized by a wired logic. The program is recorded on a recording medium (will be described later) and mechanically read by the target nucleic acid measuring apparatus 100 as needed. More specifically, on the storage unit 106 such as a ROM or an HD, a computer program which gives an instruction to the CPU in cooperation with an operating system (OS) to perform various processes is recorded. The computer program is executed by being loaded on a RAM, and constitutes a control unit in cooperation with the CPU. As an example of a target nucleic acid measuring program, the following may be utilized.

A computer program product having a computer readable medium including programmed instructions for a computer having a control unit and a storage unit,

wherein the storage unit includes:

an amplification amount detection intensity storage unit that stores a detection intensity of an amplification amount of target nucleic acid, corresponding to thermal cycle number; and

a theoretical expression storage unit that stores a theoretical expression including an environmental coefficient as an exponent parameter, a parameter of an initial template amount in the test sample, and terms for internal standard correction and baseline correction of the detection intensity of the amplification amount of the target nucleic acid, and including at least one parameter among a saturation amount upon the target nucleic acid amplification, a reaction acceleration coefficient, and a reaction inhibition coefficient, and

wherein the instructions, when executed by the control unit of the computer, cause the computer to perform:

a theoretical expression fitting step of fitting the theoretical expression to the detection intensity of the amplification amount of the target nucleic acid for each thermal cycle number stored in the storage unit; and

an initial template amount calculating step of calculating the initial template amount from the theoretical expression fitted at the theoretical expression fitting step.

The computer program may be stored in an application program server connected to the target nucleic acid measuring apparatus 100 through an arbitrary network 300. The computer program in whole or in part may be downloaded as needed.

A program which causes a computer to execute a method according to the present invention may also be stored in a computer readable recording medium. In this case, the “recording medium” includes an arbitrary “portable physical medium” such as a flexible disk, a magnet-optical disk, a ROM, an EPROM, an EEPROM, a CD-ROM, an MO, or a DVD or a “communication medium” such as a communication line or a carrier wave which holds a program for a short period of time when the program is transmitted through a network typified by a LAN, a WAN, and the Internet.

The “program” is a data processing method described in an arbitrary language or a describing method. As a format of the “program”, any format such as a source code or a binary code may be used. The “program” is not always singularly constructed, and includes a program obtained by distributing and arranging multiple modules or libraries or a program that achieves the function in cooperation with another program typified by an operating system (OS). In the apparatuses according to the embodiments, as a specific configuration to read a recording medium, a read procedure, an install procedure used after the reading, and the like, known configurations and procedures may be used.

Various databases or the like (the measurement data file 106a, the theoretical expression file 106b, the relational expression file 106c and the like) stored in the storage unit 106 are a memory device such as a RAM or a ROM, a fixed disk device such as a hard disk drive, and a storage unit such as a flexible disk or an optical disk and store various programs, tables, databases, Web page files used in various processes or Web site provision.

The target nucleic acid measuring apparatus 100 may be realized by connecting a known information processing apparatus such as a personal computer or a workstation and installing software (including a program, data, or the like) which causes the information processing apparatus to realize the method according to the present invention.

Furthermore, a specific configuration of distribution and integration of the devices is not limited to that shown in the drawings. All or some devices can be configured such that the devices are functionally or physically distributed and integrated in arbitrary units depending on various additions.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A target nucleic acid measuring apparatus comprising a storage unit and a control unit,
wherein the storage unit includes:
an amplification amount detection intensity storage unit that stores a detection intensity of an amplification amount of target nucleic acid, corresponding to thermal cycle number; and
a theoretical expression storage unit that stores a theoretical expression including an environmental coefficient as an exponent parameter, a parameter of an initial template amount in the test sample, and terms for internal standard correction and baseline correction of the detection intensity of the amplification amount of the target nucleic acid, and including at least one parameter among a saturation amount upon the target nucleic acid amplification, a reaction acceleration coefficient, and a reaction inhibition coefficient, and
wherein the control unit includes:
a theoretical expression fitting unit that fits the theoretical expression to the detection intensity of the amplification amount of the target nucleic acid for each thermal cycle number stored in the storage unit; and
an initial template amount calculating unit that calculates the initial template amount from the theoretical expression fitted by the theoretical expression fitting unit.

2. The target nucleic acid measuring apparatus according to claim 1,
wherein the terms for the internal standard correction and the baseline correction in the theoretical expression stored in the theoretical expression storage unit, include:
a term that performs the internal standard correction by dividing the detection intensity of the amplification amount of the target nucleic acid by the detection intensity of an internal standard for each thermal cycle number; and
a term that performs the baseline correction by subtracting from the term that performs the internal standard correction, a value obtained by dividing a background value of the detection intensity of the amplification amount of the target nucleic acid by a background value of the detection intensity of the internal standard.

3. The target nucleic acid measuring apparatus according to claim 1,
wherein an amplification efficiency of the nucleic acid amplification reaction is defined by the environmental coefficient and at least one parameter among the saturation amount upon the target nucleic acid amplification, the reaction acceleration coefficient, and the reaction inhibition coefficient, in the theoretical expression stored in the theoretical expression storage unit.

4. The target nucleic acid measuring apparatus according to claim 3,
wherein the theoretical expression stored in the theoretical expression storage unit, expresses that the amplification amount of the target nucleic acid derived using the terms for the internal standard correction and the baseline correction, is equal to a number obtained by subtracting the initial template amount from the product of the initial template amount and a sequence of the amplification efficiencies to each of which 1 is added for each thermal cycle number.

5. The target nucleic acid measuring apparatus according to claim 4,
wherein the theoretical expression stored in the theoretical expression storage unit is represented by the following expression:
$${N}_{0} [ {\prod }_{j = 1}^{n} \{ 1 + {ρ ( \frac{{N}_{max} - {N}_{j - 1}}{{N}_{max} + μ {N}_{j - 1}} )}^{K} \} - 1 ] = \frac{{I}_{00}}{{S}_{1}} ( \frac{{I}_{1 n}}{{I}_{0 n}} - \frac{{I}_{10}}{{I}_{00}} )$$
(wherein N₀ is the initial template amount; j is the thermal cycle number; Nⱼ is the amplification amount of the target nucleic acid at the thermal cycle number j; Nₘₐₓ is the saturation amount upon the target nucleic acid amplification; ρ is the reaction acceleration coefficient; μ is the reaction inhibition coefficient; K is the environmental coefficient; S₁ is the detection intensity per unit of the target nucleic acid; I₁ₙ is the detection intensity of the amplification amount of the target nucleic acid at the thermal cycle number n; and I₀ₙ is the detection intensity of the internal standard at the thermal cycle number n).

6. The target nucleic acid measuring apparatus according to claim 1,
wherein the theoretical expression fitting unit fits the theoretical expression using the least square method.
